(19) United States
(12) Patent Application Publication
(10) Pub. No.: US 2012/0184454 A1
(43) Pub. Date: Jul. 19, 2012

(54) GENE SIGNATURE IS ASSOCIATED WITH EARLY STAGE RECTAL CANCER RECURRENCE

(76) Inventors: Matthew F. KALADY, Cleveland Heights, OH (US); Hemant ISHWARAN, Pinecrest, FL (US)

(21) Appl. No.: 13/351,721

(22) Filed: Jan. 17, 2012

Related U.S. Application Data
(60) Provisional application No. 61/432,737, filed on Jan. 14, 2011.

(51) Int. Cl. C40B 30/04; G01N 33/566; C40B 40/06; C12Q 1/68

(52) U.S. Cl. 506/9; 435/6.12; 435/7.1; 506/16

(57) ABSTRACT

Applicants have identified a 36-gene signature that is associated with recurrence of stage I and II rectal cancers not treated with chemotherapy or radiation. This signature can be used to identify early stage rectal cancer patients that may need additional therapy beyond surgery to treat their cancer as well as identify patients that will not benefit from therapy other than surgery.

### CROSS REFERENCE TO RELATED APPLICATIONS

This application claims priority to U.S. Provisional Patent Application No. 61/432,737 filed on Jan. 14, 2011, the disclosure of which is hereby expressly incorporated by reference in its entirety.

### BACKGROUND

Despite clinical advances, rectal cancer remains a significant cause of cancer-related death. Treatment strategies and clinical outcomes are determined by cancer stage as defined by local tumor penetration and spread to lymph nodes or distant organs. Although patients with early stage rectal cancers generally enjoy excellent outcomes with surgery as the sole treatment, advanced tumors have a worse prognosis and are additionally treated with neoadjuvant or adjuvant chemotherapy and/or radiation. In spite of established treatment protocols, a significant number of early stage rectal cancer patients still develop recurrent cancer and die from their disease.

Unfortunately, there is no accurate means to predict which patients with early stage disease will suffer recurrence, so there is no way of identifying which patients should be targeted for neoadjuvant treatment or adjuvant treatment. An accurate prognostic model could identify which patients might benefit from chemotherapy and/or radiation while sparing risks for those who would not benefit. Although various molecular markers involved in colorectal cancer etiology have been identified, the process of oncogenesis and cancer metastasis is likely a complex chain of events with multiple intertwined pathways, most of which remain unknown.

The search for new factors has been boosted by development of technologies capable of high-throughput analysis such as microarrays for gene expression. Broad molecular and genetic analyses using these techniques have been performed and validated for various tumors including colorectal cancer (see for example Potti, et al. N Engl J Med 2006; 355:570-580; Rosenwald, et al., N Engl J Med 2002; 346:1937-1947; van 't Veer, et al., Nature 2002; 415:530-536; and Wang et al. J Clin Oncol 2004; 2 2:1564-1571). As reported herein a well-defined rectal cancer population is used along with microarray technology to develop a gene signature that accurately predicts recurrence or non-recurrence of early stage rectal cancer.

### SUMMARY

As disclosed herein a pattern of gene expression has been discovered that correlates with the likelihood of recurrence of cancer in rectal cancer patients who have received surgery to remove rectal cancer tumors. Altered gene expressions provide a gene signature that is predictive of recurrence or non-recurrence of cancer.

In accordance with one embodiment a method is provided for predicting the recurrence of rectal cancer in a patient treated by surgery alone. The method comprises the steps of determining a level of expression of three or more different test genes in a patient-derived biological sample. An increase or decrease in the expression levels of the test genes, as compared to the expression of a control gene, indicates that the subject suffers from or is at risk of a recurrence of rectal cancer. In one embodiment the test genes are selected from the group consisting of SALL4, MCOLN2, MS4A12, SPOCK1, LOC652113, REEP1, HNT, LOC652470, SERPINB5, CLEC4D, HTR4, ANKRD19, COL11A1, HLA-DRA, HOXB8, FAM23, MMP11, MAPK12, PIGR, FAM55D, IGF2, TCN1, LOC651751, KRT17, COMP, SELENBP1, STMN3, LOC649923, DEFB1, DAZ1 DAZ4, HLA-DRB4, TNFRSF11B, SLC39A2, SLC26A3, CEACAM7, SBP and CA9.

In one embodiment the expression of all 36 test genes noted in the preceding paragraph are analyzed using a scoring-pair approach. The scoring scoring-pair approach allows each differentially expressed gene to be scored individually as −1, +1, or zero, relative to a control gene. A non-zero value was assigned only when one could find a control gene whose expression value consistently lay between expression values for the 2 outcome groups, ensuring that differential expression was not only statistically significant but also biologically consistent. Each of these values for the 36 genes was summed to derive a score for that particular patient. So, any one patient can have a score from −36 to +36 that is associated with a particular risk. The histogram of these scores is shown in FIG. 2. Although there is overlap in the middle range of these scores, the model is particularly useful for patients scoring at the extremes of the scale, where there is a nearly 100% chance of either recurrence or non-recurrence of disease.

The present disclosure also encompasses kits comprising nucleic acid probes, antibodies for detection of one or more of the signature genes or their encoded protein or mRNA as well as other regents for detecting and quantitating the corresponding signature gene mRNA or encoded protein. In one embodiment the kit further includes a nucleic acid array comprising sequences that hybridize to one or more of the signature genes or their corresponding mRNA. In one embodiment the signature genes include SALL4, MCOLN2, SPOCK1, REEP1, HNT, SERPINB5, CLEC4D, HTR4, ANKRD19, COL11A1, HLA-DRA, FAM23, MMP11, MAPK12, PIGR, IGF2, TCN1, KRT17, STMN3, DEFB1, TNFRSF11B, SLC26A3, CEACAM7, SBP and CA9. In a further embodiment the signature genes are selected from the group consisting of HNT, HTR4, TNFRSF11B and SLC26A3. In one embodiment the kit comprises and array of at least four different nucleic acid sequences covalently linked to a solid support wherein the four different nucleic acid sequences comprise at least a 10 or 15 nucleic acid sequence that is identical to a contiguous sequence contained in four genes selected from the group consisting of HTR4, SBP, SLC26A3, and TNFSF 11B.

In accordance with one embodiment a method of determining a therapy for treating a rectal cancer patient is provided. The method entails analyzing the expression of the signature genes in a rectal cancer patient to determine the likelihood of a recurrence of cancer and adjusting therapeutic strategies based on the expression pattern of the signature genes. In one embodiment the method comprises the steps of obtaining the relative expression values of three or four genes selected from the group consisting of HTR4, SBP, SLC26A3, and TNFSF 11B, in a biological sample recovered from the patient. Typically, the biological sample will be a biopsy sample of the patient's tumor. This can also be derived from the resected surgical specimen. The detected expression of the signature genes will be determined relative to the expression of a control gene, wherein the expression pattern of the test genes indicates whether surgery alone is an acceptable therapy. In one embodiment, a relative increase in SBP and TNFSF 11B expression in combination with a relative decrease in HTR4 and SLC26A3 expression is correlated with patients that will have recurrence of cancer when treated only with surgery. Thus for these patients further treatment strategies should be considered.

### BRIEF DESCRIPTION OF THE DRAWINGS

FIG. 1 is a graph demonstrating a receiver operator characteristic (ROC) curve for tuned scoring-pair classifier. The ROC curve was calculated using test-fold data from K-fold validation (see Example 1 for details). Data are based on 1,000 independent replications.

FIG. 2 is a bar graph representing data generated from histogram analysis conducted for individual patient score values for the 36-gene signature. Each of the 36 genes in the signature was compared with a control gene and given a score of −1, 0, or +1 depending on decreased, similar, or increased expression, respectively. The 36 values for each patient were summed, allowing a possible range of scores from −36 to +36. White bars represent patients who developed recurrent disease and grey bars represent patients without recurrent disease. Data are based on test-fold data. Scores on the extremes of this histogram are highly predictive for either recurrence or non-recurrence.

FIG. 3 is a graph demonstrating the combined variable importance of genes in the signature. Individual genes are listed on the x-axis. The top line (R) represents recurrent patients and the bottom line (N) represents non-recurrent patients. Larger differences between data points of the two lines for each gene indicate a greater predictive power between recurrent and non-recurrent phenotypes.

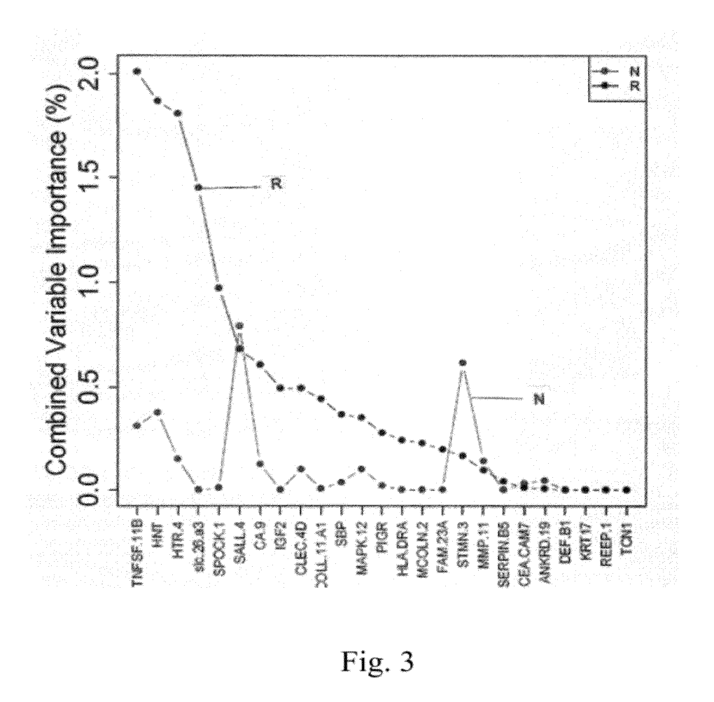

### DETAILED DESCRIPTION

### Definitions

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, “cancer” and “tumor” are synonymous terms. The term “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

As used herein, the term “gene expression” or “expression” refers to the transcription of a DNA molecule into a transcribed RNA molecule and/or the translation of the RNA transcript to produce a polypeptide. Accordingly, the expression of a gene can be detected and quantitated by measuring either its RNA transcript or the polypeptide gene product.

As used herein, an “expression pattern” is any pattern of differential gene expression. The expression pattern may relate to differences in tissue, temporal, spatial, quantity, developmental, stress, environmental, physiological, pathological, cell cycle, and chemically responsive expression patterns. As used herein, without any further modification, the term “expression pattern” relates to difference in relative expression levels.

In the context of the present invention, the phrase “control level” refers to a gene expression level detected in a control sample and includes both a normal control level and a rectal cancer control level. A control level can be a single expression pattern derived from a single reference population or from a plurality of expression patterns. For example, the control level can be a database of expression patterns from previously tested cells. A “normal control level” refers to a level of gene expression detected in a normal, healthy individual or in a population of individuals known not to be suffering from rectal cancer. A normal individual is one with no clinical symptoms of rectal cancer. Normal control can also be derived from histologically normal tissue from a patient with rectal cancer. On the other hand, a “rectal cancer control level” refers to an expression profile of rectal cancer-associated genes found in a population suffering from rectal cancer. As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a compound refers to a nontoxic but sufficient amount of the peptide to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolythic strip or sheet. The surface of such supports may be solid or porous and of any convenient shape.

The term “linked” or like terms refers to the connection between two groups. The linkage may comprise a covalent, ionic, or hydrogen bond or other interaction that binds two compounds or substances to one another.

### EMBODIMENTS

The present disclosure relates to the discovery of a subset of genes whose pattern of expression provides information regarding the prognosis of the recurrence of rectal cancer after surgery to remove tumors from a patient suffering form early stage rectal cancer (i.e., stage I and II rectal cancers). Applicants have discovered that the expression of genes selected from the group consisting of SALL4, MCOLN2, MS4A12, SPOCK1, LOC652113, REEP1, HNT, LOC652470, SERPINB5, CLEC4D, HTR4, ANKRD19, COL11A1, HLA-DRA, HOXB8, FAM23, MMP11, MAPK12, PIGR, FAM55D, IGF2, TCN1, LOC651751, KRT17, COMP, SELENBP1, STMN3, LOC649923, DEFB1, DAZ1 DAZ4, HLA-DRB4, TNFRSF11B, SLC39A2, SLC26A3, CEACAM7, SBP and CA9 have predictive value for whether a patient will experience a recurrence of cancer within 3-6 years after surgery to remove the original tumor(s).

Patients whose tumors fit the gene signature for recurrence could potentially benefit from neoadjuvant or adjuvant treatments. Conversely, patients with stage II rectal cancer whose gene profile is consistent with non-recurrent disease could possibly forgo neoadjuvant chemoradiation because total mesorectal surgery alone provides long-term cure in the majority of cases. So, directed selection of patients to receive neoadjuvant or adjuvant therapy could minimize unnecessary treatment.

In accordance with one embodiment a method for predicting the recurrence of rectal cancer in a patient treated by surgery alone is provided. The method comprises the steps of determining a level of expression of three or more different test genes in a patient-derived biological sample and analyzing the expression pattern of the genes. In one embodiment, the expression of a gene is characterized by detecting its corresponding transcribed mRNA. Alternatively, in another embodiment the expression of a gene is characterized by detecting the encoded protein gene product of the gene. Any standard technique known to those skilled in the art can be used to detect and quantitate mRNA (e.g., labeled probes, PCR, reverse transcription, microarrays) or the polypeptide gene product (e.g., antibodies, mass spec).

In accordance with one embodiment altered expression patterns of the test genes (including either an increase or decrease in expression levels), as compared to the expression of a control gene, will be indicative of whether the patient is at risk for recurrence of cancer after surgery. In one embodiment the signature genes are selected from the group consisting of SALL4, MCOLN2, MS4A12, SPOCK1, LOC652113, REEP1, HNT, LOC652470, SERPINB5, CLEC4D, HTR4, ANKRD19, COL11A1, HLA-DRA, HOXB8, FAM23, MMP11, MAPK12, PIGR, FAM55D, IGF2, TCN1, LOC651751, KRT17, COMP, SELENBP1, STMN3, LOC649923, DEFB1, DAZ1 DAZ4, HLA-DRB4, TNFRSF11B, SLC39A2, SLC26A3, CEACAM7, SBP and CA9.

One unique aspect of this signature is the paired-scoring concept. Unsupervised approaches (e.g., hierarchical clustering) tend to find statistical separation in outcomes unrelated to biology; semisupervised approaches (e.g., nearest-centroid classification) find valid biologic class separation but tend to be accurate over only select phenotype groups. Unlike these approaches, in one embodiment a strongly supervised approach was used that separated both outcome groups equally well, both statistically and biologically. To do so, a scoring-pair approach is used that allows each differentially expressed gene to be scored individually as −1, +1, or zero, relative to a control gene. A non-zero value was assigned only when one could find a control gene whose expression value consistently lay between expression values for the 2 outcome groups, ensuring that differential expression was not only statistically significant but also biologically consistent.

Applicants have identified a 36-gene signature that is associated with recurrence of stage I and II rectal cancers not treated with chemotherapy or radiation. This signature is based on tissue from the primary tumor through the use of a microarray platform.

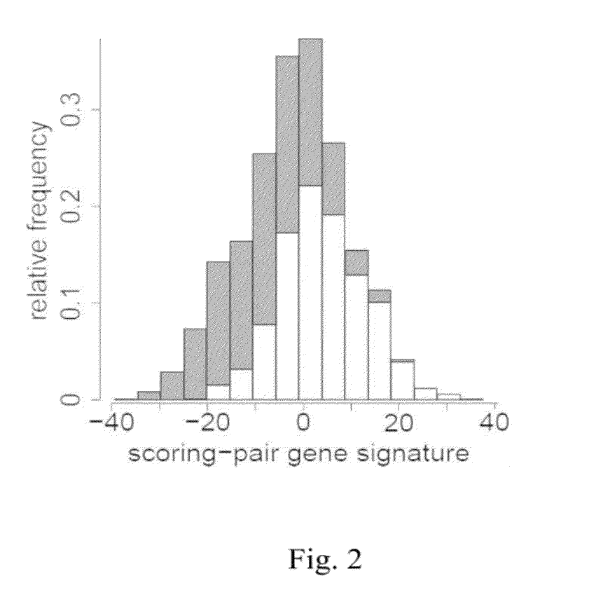

Specimens used were fresh frozen tissues (N=100). Each of these values for 36 genes, consisting of SALL4, MCOLN2, MS4A12, SPOCK1, REEP1, HNT (neurotrimin), SERPINB5, CLEC4D, HTR4, ANKRD19, COL11A1, HLA-DRA, HOXB8, FAM23, MMP11, MAPK12, PIGR, FAM55D, IGF2, TCN1, KRT17, COMP, SELENBP1, STMN3, DEFB1, DAZ1 DAZ4, HLA-DRB4, TNFRSF11B, SLC39A2, SLC26A3, CEACAM7, SBP and CA9 was summed to derive a score for that particular patient. So, any one patient can have a score from −36 to +36 that is associated with a particular risk. The histogram of these scores is shown in FIG. 2. Although there is overlap in the middle range of these scores, the model is particularly useful for patients scoring at the extremes of the scale, where there is a nearly 100% chance of either recurrence or non-recurrence of disease.

In accordance with one embodiment a method for predicting the recurrence of rectal cancer in a patient treated by surgery alone is provided. The method comprises the steps of determining a level of expression in a patient-derived biological sample of three or more different test genes, selected from the group consisting of SALL4, MCOLN2, MS4A12, SPOCK1, LOC652113, REEP1, HNT, LOC652470, SERPINB5, CLEC4D, HTR4, ANKRD19, COL11A1, HLA-DRA, HOXB8, FAM23, MMP11, MAPK12, PIGR, FAM55D, IGF2, TCN1, LOC651751, KRT17, COMP, SELENBP1, STMN3, LOC649923, DEFB1, DAZ1 DAZ4, HLA-DRB4, TNFRSF11B, SLC39A2, SLC26A3, CEACAM7 and CA9, wherein the expression of 4, 5, 10, 20 or 36 test genes of claim 1 are analyzed using a scoring-pair approach wherein a score of within 20 to 30% of the maximum score is indicative of recurrence or non-recurrence. For example, using a scoring-pair approach for 36 of the signature genes, a score of −20 to −36 is indicative that the patient will experience a non-recurrence of rectal cancer and a score of +20 to +36 is indicative that the patient will experience a recurrence of rectal cancer.

In one embodiment the relative expression of the test gene is measured by detecting mRNA of each respective test gene. The detection an measurements of the relative mRNA levels can be conducted using standard techniques known to those skilled in the art and may include amplification of the original mRNA and/or conversion to cDNA prior to measuring the relative levels of the respective gene expression. In one embodiment the detection and measurement of gene expression is conducted using a nucleic acid array. In an alternative embodiment the expression of the signature genes is detected and quantitated by detecting the protein encoded by each respective signature gene. The detection of proteins can be conducted using standard techniques including the use of antibodies for immunohistochemistry or ELISA based assays as well as by mass spectrometry.

In accordance with one embodiment the relative expression of four or more signature genes selected from the group consisting of SALL4, MCOLN2, SPOCK1, REEP1, HNT, SERPINB5, CLEC4D, HTR4, ANKRD19, COL11A1, HLA-DRA, FAM23, MMP11, MAPK12, PIGR, IGF2, TCN1, KRT17, STMN3, DEFB1, TNFRSF11B, SLC26A3, CEACAM7, SBP and CA9 is investigated to determine the likelihood of rectal cancer recurrence when surgery is the sole treatment. In a further embodiment the relative expression of the signature genes TNFRSF11B, SLC26A3, HNT, HTR4 and SPOCK1 as a group is investigated. In another embodiment the relative expression of the signature genes HNT, HTR4, TNFRSF11B and SLC26A3 as a group is investigated. In another embodiment the relative expression of the signature genes HTR4, SBP, SLC26A3, and TNFSF 11B as a group is investigated.

In one embodiment a method for detecting rectal cancer patients who will likely have a recurrence of rectal cancer if surgery alone is used as the initial treatment is provided. The method comprises the steps of obtaining a biological sample from said patient, preparing purified nucleic acids from said sample, and analyzing the expression of four test genes relative to a control gene wherein the test genes are HTR4, SBP, SLC26A3, and TNFSF 11B. In one embodiment the biological sample is tissue from a primary tumor. The analysis step may include any techniques used to quantitate the relative expression of the genes including nucleic acid hybridizations, PCR and preparation of cDNA and well as any statistical analysis to compare the relative expression of the signature genes. In accordance with one embodiment a detected increase in SBP and TNFSF 11B expression in combination with a relative decrease in HTR4 and SLC26A3 expression is correlated with patients that will have recurrence when treated only with surgical treatment. Accordingly such patients could potentially benefit from adjuvant treatments. Conversely, patients with rectal cancer whose gene profile is consistent with non-recurrent disease could possibly forgo neoadjuvant chemoradiation because total mesorectal surgery alone provides long-term cure in the majority of cases.

In accordance with one embodiment method of determining a therapeutic regimen for treating a rectal cancer patient is provided. The method comprises the steps of obtaining the relative expression values of three or four genes selected from the group consisting of HTR4, SBP, SLC26A3, and TNFSF 11B, in cancerous tissue of the patient; and comparing those expression values to the expression of a control gene, wherein an increase or decrease in the expression of the test genes indicates whether surgery alone is an acceptable therapy. More particularly, a relative increase in SBP and TNFSF 11B expression in combination with a relative decrease in HTR4 and SLC26A3 expression is correlated with patients that will have recurrence when treated only with surgery. Thus further treatment strategies should be considered for such patients. The application of a 4-gene signature using mRNA of the genes HTR4, SBP, SLC26A3, and TNFSF 11B derived from tumors embedded in paraffin provides a widely applicable test that can be derived from readily available tissues. The signature is based on a combination of the expression levels of the 4 genes. Initial validation study in independent FFPE samples demonstrates good performance as disclosed in Example 3. Each of the groups were associated with a particular phenotype; i.e. patient developed recurrent disease or did not develop recurrent disease. For the patients that were in groups 4 and 5, there was a 92% recurrence rate (11 of 12 patients). These groups represented 40% of the population tested. Of the patients who were classified as group 1 or 2, there was a 79% rate of disease-free long term survival (no recurrence). These populations represented 47% of the population. Only 4 patients (13%) fell into group 3. For this group, the 50% recurred.

As disclosed herein the signature genes were identified based on statistical methodology that identified those genes that were differentially expressed in rectal cancer cells. More particularly, in one embodiment, differentially expressed genes were identified using the automatic thresholding rule used by Bayesian Analysis of Variance for Microarrays (BAM) methodology. In accordance with one further embodiment, genes whose expression may play a role in recurrent vs. non-recurrent renal cancers can be defined based on altered expression of genes present in rectal cancer tissues. According to one embodiment, gene expression level is deemed “altered” when gene expression is increased or decreased 10%, 25%, 50% as compared to the control level. Alternatively, an expression level is deemed “increased” or “decreased” when gene expression is increased or decreased by at least 0.1, at least 0.2, at least 1, at least 2, at least 5, or at least 10 or more fold as compared to a control level. Expression is determined by detecting hybridization, e.g., on an array, of a rectal cancer-associated genes (e.g. the signature genes disclosed herein) to a gene transcript of the patient-derived tissue sample.

The present disclosure also encompasses kits comprising nucleic acid probes, antibodies for detection of one or more of the signature genes or their encoded protein or mRNA as well as other regents for detecting and quantitating the corresponding signature gene mRNA or encoded protein. In one embodiment the kit further includes a nucleic acid array comprising sequences that hybridize to one or more of the signature genes or their corresponding mRNA. In one embodiment the signature genes include SALL4, MCOLN2, SPOCK1, REEP1, HNT, SERPINB5, CLEC4D, HTR4, ANKRD19, COL11A1, HLA-DRA, FAM23, MMP11, MAPK12, PIGR, IGF2, TCN1, KRT17, STMN3, DEFB1, TNFRSF11B, SLC26A3, CEACAM7, SBP and CA9. In a further embodiment the signature genes are selected from the group consisting of HNT, HTR4, TNFRSF11B and SLC26A3. In one embodiment the kit comprises an array of at least four different nucleic acid sequences covalently linked to a solid support wherein the four different nucleic acid sequences comprise at least a 10 or 15 nucleic acid sequence that is identical to a contiguous sequence contained in four genes selected from the group consisting of HTR4, SBP, SLC26A3, and TNFRSF11B. In a further embodiment the four different nucleic acid sequences comprise at least a 10 or 15 nucleic acid sequence that is identical to a contiguous sequence contained in four genes selected from the group consisting of TNFRSF11B, SLC26A3, HNT, HTR4, SBP and SPOCK1. In another embodiment the four different nucleic acid sequences comprise at least a 10 or 15 nucleic acid sequence that is identical to a contiguous sequence contained in four genes selected from the group consisting of SBP, HTR4, TNFRSF11B and SLC26A3.

### Example 1

### Differential Gene Expression within Rectal Cancers

Methods

Patient Selection and Outcomes

The Cleveland Clinic Department of Colorectal Surgery has an IRB-approved database that collects clinical information and follow-up for colorectal cancer patients. This database was queried for patients with stage I or II rectal cancer who were treated by surgery alone. Any patients receiving pre- or postoperative chemotherapy and/or radiation were excluded to avoid the confounding influence on tumor composition and clinical outcomes. The study end-point was disease recurrence. Only patients with recurrent disease or those without recurrence and at least 3-year follow-up were included.

Time to recurrence or disease-free interval was defined as the time from the date of surgery to the date of confirmed tumor relapse for patients with recurrence, and from the date of surgery to the date of last follow-up for disease-free patients. Disease-free survival was defined as being alive without any evidence of recurrent disease as of the latest clinical follow-up. From these groups, patients with available fresh frozen tumor samples comprised the final study population. Charts were reviewed to validate the clinical endpoints of recurrent or non-recurrent cancer from the database. Basic demo-graphic, clinical, and tumor characteristics were analyzed.

### Fresh Frozen Tissue Samples

Tumor tissue was obtained according to Institutional Review Board-approved protocols using frozen tumor specimens from patients treated at the Cleveland Clinic. Tumor tissues were obtained through a dedicated tissue procurement team within the Department of Anatomic Pathology. A portion of the tumor was snap frozen and banked at −80° C. A gastrointestinal pathologist confirmed the histopathology diagnosis of each specimen independently. Specimens chosen for analysis contained at least 60% tumor cells.

RNA Isolation from Frozen Tissue Samples

RNA was extracted from fresh frozen tumor tissue. Frozen tissue blocks stored at −80° C. were cut on a microtome into 5×10 um-thick samples and resuspended in 100 uL of tissue lysis buffer, 16 uL 10% sodium dodecyl sulfate (SDS) and 80 uL Proteinase K. Samples were vortexed and incubated in a thermomixer set at 400 revolutions per minute for 3 hours at 55° C. Subsequent steps of sample processing were performed according to manufacturer's protocol. RNA samples were quantified by optical density 260/280 readings using a spectrophotometer and diluted to a final concentration of 50 ng/uL. To assure RNA quality, the mRNA of each specimen was run on a gel to assure lack of degradation before being hybridized for the microarray.

### Total Genome Gene Expression Analysis

Isolated total genome RNA was tested for total genome expression using >46,000 transcript-specific sequences on the Sentrix Human-6 Expression BeadChip (Illumina). Briefly, 100 ng of total RNA was amplified by an in vitro transcription amplification kit (Ambion) and hybridized to the platform using commercially available kits (Illumina). Illumina BeadStation 500 software was used for imaging and normalization of data.

### Statistical Analysis

Quantitative variables are summarized by mean+standard deviation or median with interquartile ranges. Categorical variables are summarized by frequency. Demo-graphic and tumor differences between recurrent and non-recurrent populations were assessed using chi-square or Fisher's exact test for categorical variables and Wilcoxon rank sum test for quantitative variables. Because recurrence occurred at various follow-up times and not all patients were observed with equal follow-up, factors associated with recurrence were best assessed using the log-rank estimates and Kaplan-Meier analyses for recurrence-free survival.

### Microarray Statistical Analysis

Microarray data (43,148 probes per sample) were background corrected and median baseline normalized using the Beadarray R-software package for Bioconductor. Normalized data were analyzed using Bayesian Analysis of Variance for Microarrays (BAM) methodology. Computations were implemented using BAMarray 2.0 software under the no-baseline option assuming unequal variances across cancer (phenotype) groups, with variance clustering set to 2 clusters. To invoke the no-baseline option, normalized data were transformed by baseline centering. For each sample (69 non-recurrent and 31 recurrent tissues), expression values were subtracted from the corresponding probeset for all patients in the opposing phenotype class. So, each probeset for the 69 non-recurrent samples had 31 baseline expression values, and each probeset for the 31 recurrent samples had 69 baseline expression values. This resulted in 4,278 observations (69×31+31×69) for each probeset, and a total of 184,587,144 (4,278×43,148) data values.

Computations were implemented on an Altix 350 Silicon Graphics multiprocessor server. A total of 52 genes from the 43,148 probes were found to be differentially expressed using the automatic thresholding rule used by BAMarray. Subsequent analyses focused on these 52 genes. The first method used to further condense the gene signature included nearest shrunken centroid classification. Using normalized expression data for the 52 genes, a nearest shrunken centroid classifier was derived. Computations were implemented using the pamr R-software package. Misclassification error rate for the classifier was estimated using 5-fold cross-validation.

A second method to develop the gene signature using a scoring-pair algorithm was derived using control genes. Candidate control genes were defined by removing the 52 differentially expressing genes, as well as all genes with BAM test statistics exceeding a nominal cut-off value, from the 43,148 probes. All Illumina specific probesets that could not be annotated using data from National Center for Biotechnology Information (http://www.ncbi.nlm.nih.gov) were also removed. This left a total of 2,325 candidate control probe sets.

Balanced K-fold validation (K=6) was used in the gene signature development. The classifier was trained using 4 folds of the data (training folds). To train the classifier, a control gene from the candidate pool was found for each of the 52 differentially expressing genes. A control gene (Cg) for a gene (g) was defined as that gene with the maximum number of expression values lying between the mean expressions for the 2 phenotype groups for g. The classifier was defined by assigning the value +1 to a gene g if the mean expression for g for the recurrent tissues was larger than Cg, otherwise it was defined to be −1. The overall score for the classifier was the sum total over the 52 genes. The trained classifier was tuned on the fifth-fold (tuning-fold) of the data. This was done by varying the number of genes in the signature. The classifier with highest area under the receiver operator characteristic (ROC) curve was chosen. The accuracy of the tuned classifier was estimated by calculating its area under the ROC curve using the sixth-fold (test-fold) of the data. This process was repeated 1,000 times independently. The final classifier was defined by using only genes that appeared more than 70% of the time with the same +1 or −1 score. This resulted in a gene signature comprising 36 genes and 36 matched control genes (“scoring-pair” signature). Accuracy of this classifier was estimated by the area under the ROC curve using the 1,000 test-fold datasets.

### Results

### Patient and Tumor Characteristics

Fresh frozen tumors from 100 patients were available to build the predictive model: 69 rectal cancers from patients with non-recurrent disease and 31 rectal cancers from patients who subsequently developed recurrence. All cancers were from pathologic early stage, node-negative, rectal cancer patients who were treated by surgical resection alone with curative intent. Eighty patients underwent low anterior resection, 18 underwent abdominoperineal resection, and 2 underwent total proctocolectomy. Patients undergoing local excision were not included in this study. No patients received preoperative chemoradiation or adjuvant treatment before recurrence. Mean follow-ups for non-recurrent and recurrent patients were 120 and 66.9 months, respectively. The median follow-up for non-recurrent patients was 104.6 months, with 25th, 50th, and 75th percentiles of 58.3, 104.6, 172.7, respectively (interquartile range 114.4 months). The mean time to recurrence was 37.1 months. There were 24 patients with distant recurrence, 6 patients with local recurrence, and 1 patient with both distant and local recurrence.

Demographics and tumor characteristics are shown in Table 1. Patients with non-recurrent cancer had higher mean and median numbers of lymph nodes evaluated than those with recurrent disease: 23 versus 16, and 20 versus 12, respectively (p=0.04, Wilcoxon rank sum test). There were 2 significant outliers in terms of number of lymph nodes evaluated. One patient with non-recurrent disease had 180 lymph nodes evaluated. One patient with recurrent disease had no lymph nodes evaluated. This case was re-reviewed by Pathology at the time of resection and still no lymph nodes were found. If these 2 outliers are removed from the data purely to evaluate the number of nodes examined, the difference is no longer significant (p=0.10). Regardless, evaluation of at least 12 lymph nodes has been shown to be accurate for staging rectal cancer and both groups met this requirement. In addition, a log-rank estimate was performed to evaluate the influence of lymph node harvest on disease-free survival in our study population using 12 as the cut-off for number of nodes evaluated. There was no significant difference in recurrence-free survival (p=0.23).

Not unexpectedly, there was a higher percentage of stage II rectal cancers among the group of patients who developed recurrence. However, this did not statistically influence recurrence-free survival in this study population based on the log-rank test (p=0.53).

TABLE 1
Table 1. Patient Demographics and Tumor

| Characteristics Variable | Non-recurrent | Recurrent | p Value |
|---|---|---|---|
| n | 69 | 31 | |
| Mean age (y), SD | 64.5 + 11 | 65.6 + 9 | 0.79 |
| Gender, male/female | 49/20 | 19/12 | 0.34 |
| Time to recurrence | NA | 37 + 26 | NA |
| (mo), mean + SD | | | |
| Median follow-up, | 105 (114) | 32 (25) | <0.001 |
| mo (IQR) | | | |
| Distance from anal | 9.3 + 3.6 | 8.7 + 4.0 | 0.47 |
| verge (em), | | | |
| mean + SD | | | |
| Tumor size (em), | 4.3 + 1.2 | 4.7 + 1.8 | 0.27 |
| mean + SD | | | |
| Lymph nodes | 23 + 23 | 16 + 11 | 0.04 |
| examined (n), | | | |
| mean + SD | | | |
| Cancer stage, n (%) | | | 0.03 |
| I | 43 (62) | 12 (39) | |
| II | 26 (38) | 19 (61) | |
| T stage, n (%) | | | 0.04 |
| T1 | 8 (12) | 0 (0) | |
| T2 | 35 (51) | 13 (42) | |
| T3 | 26 (38) | 17 (55) | |
| T4 | 0 (0) | 1 (3) | |
| Differentiation, n (%) | | | 0.34 |
| Well | 9 (13) | 1 (3) | |
| Moderately | 52 (75) | 27 (87) | |
| Poorly | 8 (12) | 3 (10) | |

IQR, interquartile range.

### Gene Expression

A total of 52 genes from the 43,148 probes were found to be differentially expressed using the automatic thresholding rule used by BAMarray. These 52 genes include: TNFRSF11B, SALL4, NR 001564, NM 203347, KRT17, IGF2, REG1A REG1B RegL, JSRP1, DEFB1, COL11A1, COL10A1, neurotrimin, SPOCK1, SFRP2, MMP11, COMP, TCN1, SERPIN B5, HOXB8, CA4, MSLN, CA4, INDO, HLA-DRB4, HLA-DRA, HBB HBD, IGJ, MMP3, CLEC4D, SELENBP1, ST6GALNAC1, FAM55D, LEFTY1, C10orf99, CEACAM7, FAM23A FAM23B, SLC26A3, PIGR, ANKRD19, STMN3, C6orf21 LY6G6D, RUNDC3A, REEP1, HTR4, MS4A12, MCOLN2, DAZ2 DAZ3, CLDN18, SLC39A2, NKX2-1, DAZ1 DAZ4 and MAPK12. Unsupervised hierarchical clustering of BAM test statistics for the 52 differentially expressing genes identified 2 distinct populations corresponding to patients with non-recurrent or recurrent rectal cancer. The first group (recurrent) comprising TNFRSF11B, SALL4, NR 001564, NM 203347, KRT17, IGF2, REG1A REG1B RegL, JSRP1, DEFB1, COL11A1, COL10A1, neurotrimin, SPOCK1, SFRP2, MMP11, COMP, TCN1, SERPIN B5, HOXB8, CA4, MSLN and the second group (non-recurrent) comprising CA4, INDO, HLA-DRB4, HLA-DRA, HBB HBD, IGJ, MMP3, CLEC4D, SELENBP1, ST6GALNAC1, FAM55D, LEFTY1, C10orf99, CEACAM7, FAM23A FAM23B, SLC26A3, PIGR, ANKRD19, STMN3, C6orf21 LY6G6D, RUNDC3A, REEP1, HTR4, MS4A12, MCOLN2, DAZ2 DAZ3, CLDN18, SLC39A2, NKX2-1, DAZ1 DAZ4 and MAPK12. BAM test statistics (1 statistic for each gene and each sample) measured distance for a patient's gene expression to the gene expression for all other patients from the alternate phenotype. The data showed consensus among the 52 genes in delineating cancer outcome status.

### Nearest Shrunken Centroid Gene Signature

In an attempt to further condense the 52 differentially expressed genes, nearest shrunken centroid classification was used (Tibshirani et al., Proc Natl Acad Sci USA 2002; 99:6567-6572). Error rates using 5-fold validation for nearest centroid classification were flat as a function of threshold-shrinkage value. This demonstrated that the 52 nearest centroid gene signature could not be improved by removing genes. Centroids for recurrent and non-recurrent data were relatively large for all genes. Five-fold validation error rate for the classifier was 29%. Error rates were significantly lower for non-recurrent data. Predicted class probabilities also showed that the nearest centroid classifier was better over non-recurrent data.

### Scoring-Pair Gene Signature

Using an alternative method, a scoring-pair algorithm, a 36-gene signature was derived. Hierarchical unsupervised clustering of the scoring-pair 36-gene signature identified 2 subsets of genes that delineated between recurrent and non-recurrent cancer groups. A complete gene list is shown in Table 2. Twenty-two genes were

TABLE 2
Genes Included in the 36-Gene Predictive Signature

| | | Expression in | |
|---|---|---|---|
| Gen Bank ID | Gene name | recurrent | Description/annotation |
| NM_000111 | SLC26A3 | Decreased | Chloride anion exchanger (Protein DRA) |
| NM_006890 | CEACAM7 | Decreased | Carcinoembryonic antigen-related cell adhesion molecule 7 |
| | | | precursor (Carcinoembryonic antigen CGM2) |
| NM_001216 | CA9 | Increased | Carbonic anhydrase 9 precursor (EC 4.2.1.1) |
| | | | (Carbonic anhydrase IX) (Carbonate |
| | | | dehydratase IX) (CA-IX) (CAIX) (Membrane |
| | | | antigen MN) (P54/58N) (Renal cell carcinoma- |
| | | | associated antigen G250) (RCC- associated |
| | | | antigenG250) (pMW1). |
| NM_002644 | PIGR | Decreased | Polymeric-immunoglobulin receptor precursor (Poly-Ig |
| | | | receptor) |
| NM_017678 | FAM55D | Decreased | Protein FAM55D precursor |
| NM_000612 | IGF2 | Increased | Insulin-like growth factor II precursor (IGF-II) (Somatomedin |
| NM_001062 | TCN1 | Increased | Transcobalamin-1 precursor (Transcobalamin I) |
| XM_940969 | LOC651751 | Decreased | |
| NM_000422 | KRT17 | Increased | Keratin |
| NM_000095 | COMP | Increased | Cartilage oligomeric matrix protein precursor (COMP) |
| NM_003944 | SELENBP1 | Decreased | Selenium-binding protein 1 (56 kDa selenium-binding |
| | | | protein) |
| NM_015894 | STMN3 | Decreased | Stathmin-3 (SCG10-like protein) |
| XM_939003 | LOC649923 | Decreased | |
| NM_005218 | DEFB1 | Decreased | Beta-defensin 1 precursor (BD-1) (Defensin) |
| NM_020420 | DAZ1 DAZ4 | Decreased | Deleted in azoospermia protein 4 |
| NM_021983 | HLA-DRB4 | Decreased | |
| NM_002546 | TNFRSF11B | Increased | Tumor necrosis factor receptor superfamily member11B |
| | | | (Osteoprotegerin) (Osteoclastogenesis inhibitory factor). |
| NM_014579 | SLC39A2 | Decreased | Zinc transporter ZIP2 (Eti-1) (6A1) (hZIP2) (Solute |
| | | | carrier family 39 member2) |
| NM_080629 | COL11A1 | Increased | Collagen alpha-1(XI) chain precursor |
| NM_019111 | HLA-DRA | Decreased | major histocompatibility complex |
| NM_024016 | HOXB8 | Increased | Homeobox protein Hox-B8 (Hox-2D) (Hox-2.4) |
| NM_001013629 | FAM23 | Decreased | Protein FAM23A, Protein FAM23B |
| NM_005940 | MMP11 | Increased | Stromelysin-3 precursor (EC 3.4.24.-) (ST3) (SL-3) (Matrix |
| | | | metalloproteinase-11) (MMP-11) |
| NM_002969 | MAPK12 | Decreased | Mitogen-activated protein kinase 12 (EC 2.7.11.24) |
| | | | (Extracellular signal-regulated kinase 6) (ERK-6) |
| | | | (ERK5) (Stress-activated protein kinase 3) (Mitogen- |
| | | | activated protein kinase p38 gamma) (MAP kinase |
| | | | p38 gamma) |
| NM_020436 | SALL4 | Increased | Sal-like protein 4 (Zinc finger protein SALL4) |
| NM_153259 | MCOLN2 | Decreased | Mucolipin-2 |
| NM_017716 | MS4A12 | Decreased | Membrane-spanning 4-domains subfamily A member 12 |
| NM_004598 | SPOCK1 | Increased | Testican-1 precursor (Protein SPOCK) |
| XM_941444 | LOC652113 | Decreased | |
| NM_022912 | REEP1 | Decreased | Receptor expression-enhancing protein 1 |
| NM_016522 | HNT | Increased | Neurotrimin precursor (hNT) |
| XM_945536 | LOC652470 | Increased | |
| NM_002639 | SERPINB5 | Increased | Serpin B5 precursor (Maspin) (Protease inhibitor 5) |
| NM_080387 | CLEC4D | Decreased | C-type lectin domain family 4 member D (C-type |
| | | | lectin superfamily |
| NM_000870 | HTR4 | Decreased | 5-hydroxytryptamine 4 receptor (5-HT-4) (Serotonin |
| | | | receptor 4) (5-HT4) |
| NM_001010925 | ANKRD19 | Decreased | |

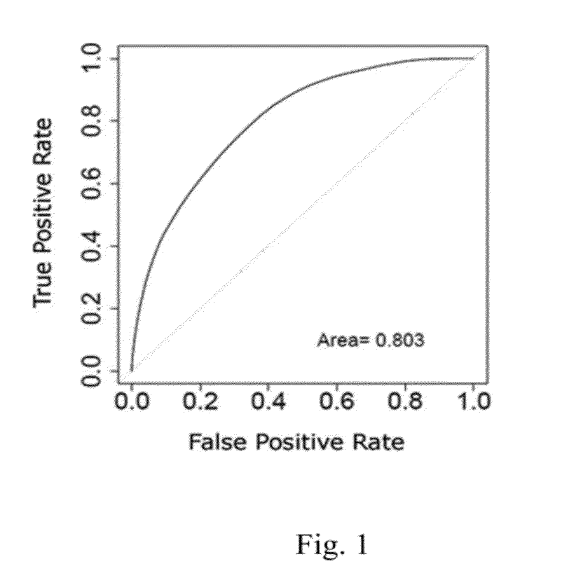

underexpressed and 14 were overexpressed in recurrent rectal cancers in relation to non-recurrent rectal cancers. The direction of expression is shown in Table 2. Overall accuracy of the 36 scoring-pair signature, as measured by area under the ROC curve, was 0.803 (FIG. 1). This was significantly better than the 52 nearest centroid gene signature. Distribution of the scores for the 36-gene signature over the 1,000 testfold datasets showed good separation between recurrent and non-recurrent data (FIG. 2).

### Discussion

This study introduces a novel means to identify early stage rectal cancer patients who are at risk for recurrence by using a predictive gene signature that can be assessed by tumor tissue analysis at the time of diagnosis or resection. The model was built on a well-characterized patient population treated by total mesorectal excision and used robust statistical methods that yield an overall accuracy of 80%.

Despite best practices, the staging system and associated treatment protocols for rectal cancer still remain flawed. Although early stage cancers are treated by surgery alone, distal disease recurrence still occurs in approximately 20% of cases. Identifying this subset of patients could allow for an opportunity to intervene with neoadjuvant or adjuvant therapy. For example, patients with stage I rectal cancer with a high probability of recurrence based on the gene expression profile could theoretically be offered neoadjuvant or adjuvant chemotherapy. The signature may also assist in a more directed therapy for stage II tumors. Current standards dictate neoadjuvant chemoradiation for stage II or III adenocarcinoma in the middle or lower third of the rectum. Patients with clinical or pathologic lymph node involvement usually also receive subsequent further adjuvant chemotherapy. However, the added benefit for adjuvant therapy for all stage II patients receiving neoadjuvant chemoradiation is controversial. Patients whose tumors fit the gene signature for recurrence could potentially benefit from adjuvant treatments. Conversely, patients with stage II rectal cancer whose gene profile is consistent with non-recurrent disease could possibly forgo neoadjuvant chemoradiation because total mesorectal surgery alone provides long-term cure in the majority of cases. Accordingly, directed selection of patients to receive neoadjuvant or adjuvant therapy could minimize unnecessary treatment.

One unique aspect of this signature is the paired-scoring concept. Unsupervised approaches (e.g., hierarchical clustering) tend to find statistical separation in outcomes un-related to biology; semisupervised approaches (e.g., nearest-centroid classification) find valid biologic class separation but tend to be accurate over only select phenotype groups. Unlike these approaches, we sought to use a strongly supervised approach that separated both outcome groups equally well, both statistically and biologically. To do so, we used a scoring-pair approach that allowed each differentially expressed gene to be scored individually as −1, +1, or zero, relative to a control gene. A non-zero value was assigned only when one could find a control gene whose expression value consistently lay between expression values for the 2 outcome groups, ensuring that differential expression was not only statistically significant but also biologically consistent. Each of these values for the 36 genes was summed to derive a score for that particular patient. So, any one patient can have a score from −36 to +36 that is associated with a particular risk. The histogram of these scores is shown in FIG. 2. Although there is overlap in the middle range of these scores, the model is particularly useful for patients scoring at the extremes of the scale, where there is a nearly 100% chance of either recurrence or non-recurrence of disease.

This work is the first to report a gene signature to predict recurrence of rectal cancer treated by surgery alone. The Memorial Sloan Kettering group studied predictors of recurrence for early stage rectal adenocarcinoma by evaluating tissue microarray expression of several known molecular markers. However, the given set of known markers did not accurately correlate with recurrence (see Hoos et al., Clin Cancer Res 2002; 8: 3841-3849) underscoring the need for better ways of predicting outcomes. Other groups have used tumor microarray platforms to predict response to preoperative radiation in Japanese rectal cancer patients (see Kawakami et al., Br J Cancer 2006; 94:593-598) and response to neoadjuvant chemoradiation in the German Rectal Cancer Trial (see Ghadimi et al., J Clin Oncol 2005; 23:1826-1838, 3233). Gene profiles in these studies are influenced by treatment interventions and cannot be extrapolated for patients treated by surgery alone.

Gene signatures have been developed for predicting recurrence of colon cancers (see Jiang et al., J Mol Diagn 2008; 10:346-354 and Barrier et al. Oncogene 2005; 24: 6155-6164. One group (Barrier) reported on 70 genes associated with recurrence of stage II and III colon cancers. None of the genes overlapped with those reported in this study, but similar genes within a common family such as insulin-like growth factor and tumor necrosis factor were found to be increased in recurrent patients in both Barrier's work and this study. A multi-institutional group (Jiang et al) reported a 7-gene signature to predict stage II colon cancer recurrence. Similarly, there was no overlap with the current study gene signature. These findings are not surprising due to genetic and molecular differences between colon and rectal cancers, heterogeneity of patients used between the studies, and differences in methodology in developing the signatures. Microarray technology is increasingly being used to identify and define genes associated with subclasses of disease.

We included a stringently defined patient population of pathologically defined stage I and II rectal cancer patients who were treated by formal resection (no local excision). Patients receiving neoadjuvant therapy were excluded to avoid the influence of medical treatment on gene expression or recurrence. All operations were done with curative intent and performed by colorectal surgeons at a tertiary care center using techniques of vessel high ligation and sharp total mesorectal excision.

In addition to identifying a gene signature, this work has identified individual genes that may be important to understanding the biologic process of cancer. Not all genes have a known biologic process nor are they linked to cancer. However, multiple genes in the signature are involved with cell adhesion and signaling, cellular proliferation, angiogenesis, and apoptosis, among others.

Downregulation of CEA cellular adhesion molecule-7 (CEACAM-7), a regulator of normal cellular differentiation, has been demonstrated in aberrant crypt foci and adenomas. Large decreases, as seen in this signature, could be associated with more aggressive disease. Loss of selenium-binding protein 1 (SELENBP1), which is de-creased in recurrent patients, is associated with a worse overall prognosis for stage II and III colorectal cancer patients. Expression of collagen matrix protein COL11A1 is seen in adenomas and sporadic colon cancers, but not normal colonic epithelium. This gene is overly expressed in recurrent patients compared with non-recurrent patients in this study. Matrix metalloproteinase 11 (MMP11), a protein instrumental in degradation of extracellular matrix and whose increased expression portends metastatic disease, is elevated in our signature for recurrent disease patients, as might be expected.

### Example 2

### Feasibility and Applicability of Signature to RT-qPCR Platform

We validated and evaluated the genes identified in the signature by measuring gene expression in samples using quantitative RT-qPCR. We randomly chose 20 recurrent and 20 non-recurrent frozen samples that were used to develop the array-based signature and isolated mRNA from these samples. Of the 36 loci identified in the signature, 4 did not have corresponding genes according to published maps. Of the remaining 32 genes, we derived meaningful expression data from 25. For the seven that did not work, the expression level was too low to be determined, despite positive controls that indicated the experiment worked. For the 25 genes, the most informative that distinguished the outcome of recurrent or non-recurrent phenotype were identified using random forest classification, a non-parametric model-free machine learning technique (Breiman, 2001). Variable importance (VIMP) for genes is shown in FIG. 4. This shows predictiveness of each gene using bootstrap cross-validation.

### Determining Outcome by Gene Expression Algorithm

To identify a simple gene signature from the RT-qPCR data, we used CART (classification and regression tree) methodology (Breiman et al, 1984) using the top genes identified from the random forest analysis. The optimal tree depth was determined using 10-fold validation. A CART analysis was applied to each of 4 independent RT-qPCR replicated experiments. Based on the tree “if-than” flowchart, patients can be classified into recurrent or non-recurrent phenotype based on gene expression. Although the overall error rate for this simple classifier is 40%, we identified two subgroups where perfect classification was achieved using a 3 gene-signature rule.

### Example 3

### Transition/Application of a 4-Gene Signature Using mRNA Derived from Tumors Embedded in Paraffin

In order to develop a more widely applicable platform, we attempted to validate the signature using mRNA derived from paraffin-embedded tissues. A four-gene signature was tested including the genes: TNFRSF.11B, HTR.4, SBP, and slc.26.a3 as identified from our preliminary CART analyses. Tumors from 30 new patients not included in the initial model were used as a test set to assess its accuracy to predict the test data phenotype.

Initial validation study in independent FFPE samples demonstrates good performance. Specifically, mRNA was isolated from tumors of 30 patients and tested for gene expression of the 4 genes. Based on expression of the 4 genes, the test population segregated itself into 5 main groups. Each of the groups were associated with a particular phenotype; i.e. patient developed recurrent disease or did not develop recurrent disease. For the patients that were in groups 4 and 5, there was a 92% recurrence rate (11 of 12 patients). These groups represented 40% of the population tested. Of the patients who were classified as group 1 or 2, there was a 79% rate of disease-free long term survival (no recurrence). These populations represented 47% of the population. Only 4 patients (13%) fell into group 3. For this group, the 50% recurred.

### Experimental Methods

### Patient Selection

The Cleveland Clinic Department of Colorectal Surgery has an IRB-approved database that collects clinical information and follow-up for colorectal cancer patients. This database was queried for patients with stage I or II rectal cancer who were treated by surgery alone. Any patients receiving pre- or post-operative chemotherapy and/or radiation were excluded to avoid the confounding influence on tumor composition and on clinical outcome. Any patients that were used to develop the initial gene signature model were excluded. Patients from this query were then further categorized into those that developed tumor recurrence or those that were alive and without evidence of tumor recurrence with at least 5 years clinical follow-up. For patients that developed a recurrence, only patients with at least 12 lymph nodes examined histologically and those with negative distal and circumferential resection margins were be included to limit technical influence on disease recurrence. Patients with cancer arising within hereditary cancer syndromes or inflammatory bowel disease were excluded.

From these groups, patients with available paraffin-embedded rectal cancer specimens were included. Patient charts and electronic records were reviewed to validate the clinical endpoints of recurrent or non-recurrent cancer from the database. Basic demographic, clinical, and tumor characteristics were also be collected and analyzed.

In our pilot RT-qPCR study we identified two subgroups of patients based on a 3-gene signature (TNFSF.11B, HTR.4 and slc.26.a3) over which the two phenotypes were separated with 0% error rate over 4 independent RT-qPCR replicates. These 2 subgroups comprised 60% of our population. Therefore, expecting that 40% of patients will not fall into one of these two groups, and assuming that the 3-gene signature was no better than our overall classifier with 40% error rate, then to identify a separation in the phenotypes having 90% accuracy (far less than the 100% observed) at an alpha=5% one-sided type-I error rate and with 95% power (beta=5%), a sample size of n=31 was needed.

RNA Isolation from FFPE Tumor Tissue

RNA was isolated using the RecoverAll™ Total Nucleic Acid Isolation Kit (Ambion, Austin, Tex.). FFPE blocks were sectioned on a standard microtome into 4 20-μm sections and the isolation will be completed according to the manufacturer's protocol. Briefly, tissue samples were deparaffinized in xylene and then subjected to a protease digestion. The RNA was then bound to a glass-fiber filter, washed, subjected to DNase treatment, and finally eluted off the column. RNA quantity and integrity were assessed on a NanoDrop (Thermo Scientific, Wilmington, Del.). RNA stocks were stored at −80° C. in multiple aliquots to avoid multiple freeze-cycles, and working dilutions were made in RNase free water as necessary.

Quantitative Real-Time PCR Analysis and Scoring Gene Expression Levels

A one-step quantitative RT-qPCR reaction was performed for each sample using pre-made TaqMan® Gene Expression Assays (Applied Biosystems, Foster City, Calif.) on the ABI Prism 7900HT Sequence Detection System. Ten-microliter reactions were performed in triplicate, and all samples were normalized to an several endogenous controls. RNA from normal, non-malignant rectal tissue were be assayed for each gene and used as the calibrator sample. SDS 2.2.2 software and the 2-ΔΔCt equation were employed to calculate a relative quantity (RQ) value for each gene of interest in each tumor sample. The RQ value is defined as the amount of target gene in the tumor sample relative to the amount in the normal calibrator sample. The validation analysis was based on the RQ values. These are the dependent “x-variables” used in the analysis.

### Immunohistochemistry Analysis for Protein Expression

Genes in the above model will further be tested for corresponding protein expression in the tumors by immunohistochemistry. Primary and secondary antibodies are commercially readily available and specifics will depend on which genes are rendered significant. This has been confirmed for the top 4 genes to be tested. Expression will be measured as no change, increased, or decreased according to intensity of stain and interpreted by a gastrointestinal pathologist. Normal colon will serve as the control tissues for immunohistochemisty staining and samples classified as increased, decreased, or no change. We anticipate some proteins will be expressed in the same direction compared to normal (i.e., a recurrent and non-recurrent tumors may both have increased expression compared to normal tissues). In this situation, a Pathology-based graded scoring system will be used to assess the degree of expression.

1. A method for predicting the recurrence of rectal cancer in a patient treated by surgery alone, said method comprising the steps of determining a level of expression of three or more different test genes in a patient-derived biological sample, wherein an increase or decrease in said expression levels of the test genes, as compared to the expression of a control gene, indicates that said subject is at risk of a recurrence of rectal cancer, wherein said test genes are selected from the group consisting of SALL4, MCOLN2, MS4A12, SPOCK1, LOC652113, REEP1, HNT, LOC652470, SERPINB5, CLEC4D, HTR4, ANKRD19, COL11A1, HLA-DRA, HOXB8, FAM23, MMP11, MAPK12, PIGR, FAM55D, IGF2, TCN1, LOC651751, KRT17, COMP, SELENBP1, STMN3, LOC649923, DEFB1, DAZ1 DAZ4, HLA-DRB4, TNFRSF11B, SLC39A2, SLC26A3, CEACAM7, SBP and CA9.

2. The method of claim 1 wherein the expression of 36 test genes of claim 1 are analyzed using a scoring-pair approach, and a score of −20 to −36 is indicative that the patient will experience a non-recurrence of rectal cancer and a score of +20 to +36 is indicative that the patient will experience a recurrence of rectal cancer.

3. The method of claim 2 wherein the relative expression of the test gene is measured by detecting mRNA of each respective test gene.

4. The method of claim 3, wherein said detection is carried out on a nucleic acid array.

5. The method of claim 2 wherein the relative expression of the test gene is measured by detecting a protein encoded by each respective test gene.

6. The method of claim 1 wherein the test genes are selected from the group consisting of SALL4, MCOLN2, SPOCK1, REEP1, HNT, SERPINB5, CLEC4D, HTR4, ANKRD19, COL11A1, HLA-DRA, FAM23, MMP11, MAPK12, PIGR, IGF2, TCN1, KRT17, STMN3, DEFB1, TNFRSF11B, SLC26A3, CEACAM7, SBP and CA9.

7. The method of claim 1 wherein the test genes are selected from the group consisting of HNT, HTR4, TNFRSF11B and SLC26A3.

8. The method of claim 1 wherein the test genes are selected from the group consisting of HTR4, SBP, SLC26A3, and TNFRSF 11B.

9. A method for detecting rectal cancer patients who will likely have a recurrence of rectal cancer if surgery alone is used as the initial treatment, said method comprising the step of measuring the expression of four test genes relative to a control gene in a biological sample obtained from said patient, wherein the test genes are HTR4, SBP, SLC26A3, and TNFSF 11B, and an increase in SBP and TNFSF 11B expression in combination with a relative decrease in HTR4 and SLC26A3 expression is correlated with patients that will have recurrence when treated only with surgical treatment.

10. The method of claim 9 wherein the biological sample is tissue from a primary tumor.

11. The method of claim 10 wherein the relative expression of the test gene is measured by detecting mRNA of each respective test gene.

12. The method of claim 11, wherein said detection is carried out on a nucleic acid array.

13. The method of claim 9 wherein the relative expression of the test gene is measured by detecting a protein encoded by each respective test gene.

14. A method of determining a therapy for treating a rectal cancer patient, said method comprising the steps of obtaining the relative expression values of three or four genes selected from the group consisting of HTR4, SBP, SLC26A3, and TNFSF 11B, in cancerous tissue of the patient; and comparing those expression values to the expression of a control gene, wherein the expression pattern of the test genes indicates whether surgery alone is an acceptable therapy.

15. The method of claim 14 wherein a relative increase in SBP and TNFSF 11B expression in combination with a relative decrease in HTR4 and SLC26A3 expression is correlated with patients that will have recurrence when treated only with surgery.

16. An array of at least four different nucleic acid sequences linked to a solid support, wherein the nucleic acid sequences comprise a 12 nucleic acid sequence that is identical to a contiguous sequence contained a gene selected from the group consisting of SALL4, MCOLN2, MS4A12, SPOCK1, LOC652113, REEP1, HNT, LOC652470, SERPINB5, CLEC4D, HTR4, ANKRD19, COL11A1, HLA-DRA, HOXB8, FAM23, MMP11, MAPK12, PIGR, FAM55D, IGF2, TCN1, LOC651751, KRT17, COMP, SELENBP1, STMN3, LOC649923, DEFB1, DAZ1 DAZ4, HLA-DRB4, TNFRSF11B, SLC39A2, SLC26A3, CEACAM7, SBP and CA9,

17. The array of claim 16 wherein the nucleic acid sequences comprise a 12 nucleic acid sequence that is identical to a contiguous sequence contained a gene selected from the group consisting of SALL4, MCOLN2, SPOCK1, REEP1, HNT, SERPINB5, CLEC4D, HTR4, ANKRD19, COL11A1, HLA-DRA, FAM23, MMP11, MAPK12, PIGR, IGF2, TCN1, KRT17, STMN3, DEFB1, TNFRSF11B, SLC26A3, CEACAM7, SBP and CA9,

18. The array of claim 16 wherein the nucleic acid sequences comprise a 12 nucleic acid sequence that is identical to a contiguous sequence contained a gene selected from the group consisting of TNFRSF11B, SLC26A3, HNT, HTR4, SBP and SPOCK1,

19. The array of claim 16 wherein the nucleic acid sequences comprise a 12 nucleic acid sequence that is identical to a contiguous sequence contained a gene selected from the group consisting of HTR4, SBP, SLC26A3, and TNFRSF11B.
